(19) United States
(12) Patent Application Publication
(10) Pub. No.: US 2014/0259191 A1
(43) Pub. Date: Sep. 11, 2014

(54) FISH ANESTHETIC AND METHOD

(71) Applicant: Dani Neifeld, Moshav Bney-Yehuda (IL)

(72) Inventor: Dani Neifeld, Moshav Bney-Yehuda (IL)

(73) Assignee: AQUA-MOR TECHNOLOGIES LTD., Petach Tikva (IL)

(21) Appl. No.: 14/240,744

(22) PCT Filed: Aug. 22, 2012
(86) PCT No.: PCT/IB2012/054246

§ 371 Date: Feb. 25, 2014

Related U.S. Application Data
(60) Provisional application No. 61/527,163, filed on Aug. 25, 2011.

(51) Int. Cl. A61K 31/045

(52) U.S. Cl. CPC A61K 31/045
USPC 800/8; 514/739

(57) ABSTRACT

This invention relates to a method of sedating, anaesthetising or euthanising an aquatic organism comprising the step of contacting said organism with linalool, a compositions, a body of water containing thereof and a method of transporting an aquatic organism.

### RELATED APPLICATIONS

This application claims the benefit of U.S. Provisional Application No. 61/527,163, the contents of which are incorporated herein by reference.

### FIELD

This invention relates to improved methods for sedating, anaesthetising and/or euthanising aquatic organisms and to compositions for use in such methods.

### BACKGROUND

The practice of catching fish or other aquatic organisms usually involves the organisms undergoing some stress. The organisms commonly associate capture with predation and therefore struggle to escape immobilization. This struggle can have a major impact on the post-mortem quality of the tissue of the organism depending upon its duration and at the pre-mortem physical condition of the organism (Lowe, T. E.; Ryder, J. M.; Carragher, J. F.; Wells, R. M. G. 1993: Flesh quality in snapper, Pagrus auratus, affected by capture stress. Journal of Food Science 58: 770-773; and Jerrett, A. R.; Stevens, J.; Holland, A. J. 1995: Tensile properties of rested and exhausted chinook salmon (Oncorhynchus tshaivytscha) white muscle. In press. Journal of Food Science (USA)).

In aquaculture, the cultured organisms are usually individually handled during their life cycle. With excitable fish species such as Chinook Salmon (Oncorhynchus tshawytscha), great care must be taken to ensure that the animals are not bruised, scaled or in any way disfigured or damaged during handling. A natural, undamaged appearance is often a critical factor in determining the final sale price of the fish.

To achieve optimum product quality during harvesting, the organisms must be maintained in a calm state. One approach which has been investigated is the use of anaesthetics during harvesting. Anaesthetics such as MS-222, 2-phenoxyethanol, benzocaine and the sedatives etomidate and metomidate (Kreiberg, H. 1992: Metomidate Sedation Minimises Handling Stress in Chinook Salmon Bulletin of the Aquacultural Association of Canada 92-3: 52-54) have been used to minimise damage during handling but their potential residual toxicity to (or misuse by) humans prevents their use during harvesting. Consequently the use of some of these materials must be discontinued at least 21 days prior to harvesting.

Non-toxic non-chemical anaesthesia has also been investigated. Commonly used non-toxic alternatives such as cold anaesthesia (Mittal, A. K. and Whitear, M. 1978: A note on cold anaesthesia of poikilotherms. Journal of Fish Biology: 519-520) or carbonic acid anaesthesia (Post, G. 1979: Carbonic Acid Anaesthesia for Aquatic Organisms. The Progressive Fish Culturist 41(3): 142-144) do induce anaesthesia but can also cause considerable trauma in the process. They are accordingly not appropriate for use in harvesting if the quality of the post-mortem flesh is to be maintained as near pre-mortem as is possible.

It is therefore apparent that a need exists for a readily available food grade anaesthetic having low or no toxicity suitable for use inter alia in the harvesting of aquatic organisms. The ideal chemical anaesthesia for harvesting would be cost-effective, have low or non-irritant qualities and be suitable for use with animals intended for human consumption.

### BRIEF DESCRIPTION

Accordingly, in accordance with an embodiment of the invention there is provided a method of sedating, anaesthetising or euthanising an aquatic organism comprising the step of contacting said organism with a compound of the formula

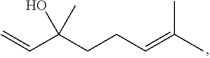

viz. linalool.

There is also provided, in accordance with an embodiment of the invention, a method of harvesting an aquatic organism while substantially retaining its pre-mortem flesh quality, comprising the step of contacting said organism with a compound of the formula

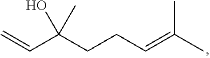

viz. linalool.

There is also provided, in accordance with an embodiment of the invention, a method of transporting an aquatic organism in a live or pre-rigor state comprising the steps of: contacting the organism to be transported with a compound of the formula

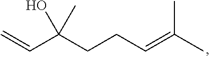

viz. linalool, in order to induce a sedated, anaesthetised or pre-rigor state in said organism; and
transporting said organism while in said sedated, anaesthetised or pre-rigor state.

In each of the above-recited methods, the compound may be (S)-(+)-linalool, viz.

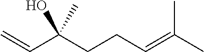

(CAS Registry Number 126-90-9), (R)-(−)-linalool, viz.

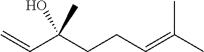

(CAS Registry Number 126-91-0), or a mixture of both (S)- and (R)-linalool, including a racemic mixture (CAS Registry number 78-70-6).

The active compound(s) ((S)- and/or (R)-linalool) may be present in a form which it is substantially free of other terpene alcohols, i.e. in a form in which at least 90 wt. % of all terpene alcohols present are linalool. The active compound(s) may be present in admixture with one or more additional food-grade aquatic sedative, anaesthetic and/or euthanising agents such as eugenol and iso-eugenol; alternatively, the linalool may be provided as at least 90 wt. % of the active ingredient, as at least 92 wt. % of the active ingredient, as at least 94 wt. % of the active ingredient, as at least 95 wt. % of the active ingredient, as at least 96 wt. % of the active ingredient, as at least 97 wt. % of the active ingredient, as at least 98 wt. % of the active ingredient, as at least 99 wt. % of the active ingredient, or as substantially 100 wt. % of the active ingredient.

The compound(s) or the admixture will usually be in solution. The active compound(s) may also be present with a surfactant. The active compound(s) may also be provided as part of a composition as described hereinbelow.

In some embodiments, the aquatic organism is present in water, and the linalool concentration is at least 5 ppm. In some embodiments, the linalool concentration is at least 6 ppm. In some embodiments, the linalool concentration is at least 7 ppm. In some embodiments, the linalool concentration is at least 8 ppm. In some embodiments, the linalool concentration is at least 9 ppm. In some embodiments, the linalool concentration is at least 10 ppm. In some embodiments, the linalool concentration is at least 15 ppm. In some embodiments, the linalool concentration is at least 20 ppm. In some embodiments, the linalool concentration is at least 25 ppm. In some embodiments, the linalool concentration is at least 30 ppm. In some embodiments, the linalool concentration is at least 35 ppm. In some embodiments, the linalool concentration is at least 40 ppm. In some embodiments, the linalool concentration is at least 45 ppm. In some embodiments, the linalool concentration is at least 50 ppm. In some embodiments, the linalool concentration is at least 60 ppm. In some embodiments, the linalool concentration is at least 75 ppm. In some embodiments, the linalool concentration is at least 100 ppm. In some embodiments, the concentration is not more that 500 ppm. In some embodiments, the linalool concentration is not more than 400 ppm. In some embodiments, the linalool concentration is not more than 300 ppm. In some embodiments, the linalool concentration is not more than 200 ppm. In some embodiments, the linalool concentration is not more than 150 ppm. In some embodiments, the linalool concentration is not more than that 100 ppm.

In some embodiments, the aquatic organism is contacted with linalool for a period of at least 1 minute. In some embodiments, the contact is for at least 2 minutes, at least 3 minutes, at least 5 minutes, at least 10 minutes, at least 15 minutes, at least 20 minutes. In some embodiments, the contact is for at least 30 minutes, at least 40 minutes, at least 50 minutes, at least 60 minutes, at least 120 minutes, or at least 180 minutes. In some embodiments, the contacting is for a period of not more than 180 minutes. In some embodiments, the contact is for not more than 120 minutes, not more than 60 minutes, not more than 50 minutes, not more than 40 minutes, not more than 35 minutes, not more than 30 minutes, not more than 25 minutes, not more than 20 minutes, not more than 15 minutes, not more than 10 minutes or not more than 5 minutes.

In accordance with an embodiment of the invention, there is also provided an active composition suitable for use as an aquatic sedative, anaesthetic or euthanising agent which comprises, in admixture, an effective amount of a compound of the formula

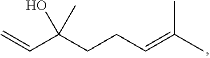

viz. linalool, and an effective amount of a solvent or surfactant. The solvent may be a polar protic or polar aprotic solvent. Examples of polar protic solvents are water, alcohols (e.g. ethanol, isopropanol), polyols (e.g. glycerol, sorbitol, propylene glycol), carboxylic acids (such as acetic acid). Examples of polar aprotic solvents are carbonyl compounds (e.g. ketones such as acetone; aldehydes such as pentanal; esters such as ethyl acetate), dimethylsulphoxide, dimethylformamide, and acetonitrite. Mixtures, blends and modifications of the aforementioned solvents are also contemplated, as will be appreciated by those skilled in the art

The composition can include one or more additional food-grade aquatic sedative, anaesthetic and/or euthanising agents, e.g. eugenol and/or iso-eugenol.

Optionally, the composition further includes a surfactant. Compositions containing the linalool may also contain an anti foam ingredient, i.e. one or more compounds used to increase the surface tension of water such as fatty acids, alcohols and polyols, silicones as well as mixtures, blends and modifications thereof as will be appreciated by those skilled in the art. The compositions may also include a preservative, i.e. a compound used to prevent, inhibit or destroy the viability, growth or propagation of micro-organisms such as bacteria, yeasts or moulds by chemical or physical means. Examples of suitable compounds for use as preservatives are formaldehyde or formaldehyde donors such as 1,3-dimethylol-5,5-dimethylhydantoin, imidiazolidinyl urea; esters of p-hydroxybetrzoic acid; alcohols and polyols such as ethanol, benzyl alcohol, pentylene glycol; and carboxylic acids such as benzoic or sorbic acid. Additional examples are compounds that increase the osmotic pressure such as sugars, glycerol, and sodium chloride. Mixtures, blends and modifications of the preservatives may also be employed, as will be appreciated by those skilled in the art. Further reference to the diverse range of possible solvents, anti:-foam agents, preservatives and surfactants can be found in standard reference books used by those skilled in the art, such as International Cosmetic Ingredient Dictionary and Handbook, 4-Volume Set, Twelfth Edition 2008, Personal Care Products Council.

The concentration of linalool in the composition may vary from very low, e.g. 0.05 wt. % or 0.5 wt. %, to fairly high, e.g. 93 wt. %. Typically the remainder of the composition will be water and one or more surfactants, although it is possible to formulate the linalool in a way that does not include water (see e.g. example 28 below). In some embodiments, the composition contains 50 wt. % linalool, 10-45 wt. % surfactant, and 40-5 wt. % solvent. In some embodiments, the composition contains 5 wt. % linalool, 5-50 wt. % surfactant; and 90-45 wt. % solvent. It will be appreciated that in use to anesthetize fishes, a relatively small amount of linalool is required generally on the order of 50-100 ppm in the water containing the fishes, and, as shown in the examples, in same cases even lower, depending on the species involved and the conditions of anesthetization—and thus, in some embodiments, the composition will be packaged with a higher linalool concentration than is necessary for use, so that the end-user will dilute the composition prior to administration to fishes. In other embodiments, the composition may be packaged so that it requires little or no dilution prior to use. The linalool may be present in admixture with one or more additional food-grade aquatic sedative, anaesthetic and/or euthanising agents such as eugenol and iso-eugenol; alternatively, the linalool may be provided as at least 90 wt. % of the active ingredient, as at least 92 wt. % of the active ingredient, as at least 94 wt. % of the active ingredient, as at least 95 wt. % of the active ingredient, as at least 96 wt. % of the active ingredient, as at least 97 wt. % of the active ingredient, as at least 98 wt. % of the active ingredient, as at least 99 wt. % of the active ingredient, or as substantially 100 wt. % of the active ingredient in the composition.

Thus, for example, in one embodiment, a formulation containing 93 wt. % linalool, 5 wt. % ethoxylated castor oil (Alkamuls 14R, CAS registry no. 61791-12-16), and 2 wt. % Amersil AF80 (an anti-foaming agent that contains glycols and is available from Ametech srl, Via Matteoti 62, 20092 Cinisello Balsamo, Italy) may be prepared and used to anesthetize fishes, for example by adding to water containing the fishes to be anesthetized to amount of about 0.006 wt. %, i.e. to a linalool concentration in the water of about 5.5 ppm. Similarly, a formulation containing 16.6 wt. % linalool, 78.3 wt. % propylene glycol, 5 wt. % ethanol and 0.1% Amersil AF80 may be prepared and used to anesthetize fishes, for example by adding to water containing the fishes to be anesthetized to amount of about 0.056 wt. %, i.e. to a linalool concentration in the water of about 55.6 ppm.

In some embodiments, the linalool is (S)-linalool. In some embodiments the linalool is (R)-linalool. In some embodiments the linalool is a mixture of (S)- and (R)-linalool.

In some embodiments, the surfactant is an ethoxylated castor oil.

In some embodiments the composition may include a solvent. In some embodiments the solvent is a polyethylene glycol which is PEG 400.

In accordance with an embodiment of the invention, there is provided a method of sedating, anaesthetising or euthanising an aquatic organism comprising the step of contacting said organism with linalool at a sufficient concentration and for a sufficient time to achieve said sedating, anaesthetising or euthanising. In some embodiments, the linalool is provided as part of a composition as described above.

As will be appreciated by persons skilled in the art, for a given type of aquatic organism of a given size, there is an inverse (though not necessarily linear) relationship between the concentration of linalool in the water and the amount of time the organism needs to be exposed to the linalool in order to achieve sedation or anesthetization, and similarly there is an inverse (though not necessarily linear) relationship between the linalool concentration and the amount of time an organism may be exposed to the linalool in order before death occurs. Thus, for example, in one case, fish left for 50 minutes in 75 ppm linalool, or left for 40 minutes in 100 ppm linalool, did not survive, but the same fish were effectively anesthetized if left for five minutes in 100 ppm linalool. On the other hand, the same fish were unaffected by exposure to 25 ppm linalool, even for long periods of time.

In accordance with an embodiment of the invention, there is provided a method, which method comprises harvesting a linalool-sedated, linalool-anesthetized or linalool-euthanized aquatic organism. In some embodiments the method comprises contacting the organism to be harvested with linalool at a concentration and for a time sufficient to induce a sedated, anaesthetised or euthanised state in said organism; and harvesting said organism while in said sedated, anaesthetised or euthanised state. In some embodiments, the linalool is provided as part of a composition as described above.

In accordance with an embodiment of the invention, there is provided a method of transporting an aquatic organism in a live or pre-rigor state comprising the steps of: contacting the organism to be transported with linalool at a concentration and for a time sufficient to induce a sedated, anaesthetised or pre-rigor state in said organism; and transporting said organism while in said sedated, anaesthetised or pre-rigor state. In some embodiments, the linalool is provided as part of a composition as described.

There is also provided, in accordance with an embodiment of the invention, a container containing water, an aquatic organism, and linalool. In some embodiments the linalool is present in a concentration sufficient to sedate or anesthetize the aquatic organism. In some embodiments, the linalool concentration is at least 5 ppm. In some embodiments, the linalool concentration is at least 6 ppm. In some embodiments, the linalool concentration is at least 7 ppm. In some embodiments, the linalool concentration is at least 8 ppm. In some embodiments, the linalool concentration is at least 9 ppm. In some embodiments, the linalool concentration is at least 10 ppm. In some embodiments, the linalool concentration is at least 15 ppm. In some embodiments, the linalool concentration is at least 20 ppm. In some embodiments, the linalool concentration is at least 25 ppm. In some embodiments, the linalool concentration is at least 30 ppm. In some embodiments, the linalool concentration is at least 35 ppm. In some embodiments, the linalool concentration is at least 40 ppm. In some embodiments, the linalool concentration is at least 45 ppm. In some embodiments, the linalool concentration is at least 50 ppm. In some embodiments, the linalool concentration is at least 60 ppm. In some embodiments, the linalool concentration is at least 75 ppm. In some embodiments, the linalool concentration is at least 100 ppm. In some embodiments, the container contains linalool in a concentration of not more that 500 ppm. In some embodiments, the linalool concentration is not more than 400 ppm. In some embodiments, the linalool concentration is not more than 300 ppm. In some embodiments, the linalool concentration is not more than 200 ppm. In some embodiments, the linalool concentration is not more than 150 ppm. In some embodiments, the linalool concentration is not more than that 100 ppm. In some embodiments the container further contains a surfactant. In some embodiments the container further contains a solvent. In some embodiments, the pH of the water is at least 5.5. In some embodiments the pH is at least 6.0. In some embodiment the pH is at least 6.5. In some embodiments the pH is as least 7.0. In some embodiments the pH is at least 7.5. In some embodiments the pH is at least 8.0. In some embodiments the pH is not more than 8.5. In some embodiments the pH is not more than 8.0. In some embodiments the pH is not more than 7.5. In some embodiments the pH is not more than 7.0. In some embodiments the pH is not more than 6.5. In some embodiments the pH is not more than 6.0. In some embodiments the temperature of the water in the container is in the range of 40° F.-75° F. (−4° C.-24° C.); in some embodiments the range is 65° F.-75° F. In some embodiments the temperature of the water in the container is in the range of 72° F.-85° F.; in some embodiments the range is 75° F.-85° F.

While the present invention is broadly defined above, it will of course be appreciated by those persons skilled in the art that it is not limited thereto but that it also includes embodiments of which the following description provides examples.

### DETAILED DESCRIPTION

The aquatic organisms to which the methods of the present invention may be applied are the so-called primary aquatic organisms which are cold blooded animals living in water and respiring dissolved oxygen, e.g. members of the class Chondrichthyes, members of the Superclass Osteichthyes. In some embodiments, the methods disclosed herein may be utilized with organism that from an economic point of view are valuable, high-grade marketable organisms. Examples of such organisms include those belonging to the class Chondrichthyes or Superclass Osteichthyes such as salmon, trout, char, ayu, carp, crucian carp, goldfish, roach, whitebait, eel, conger eel, sardine, flying fish, sea bass, sea bream, parrot bass, snapper, mackerel, horse mackerel, tuna, bonito, yellowtail, rockfish, fluke, sole, flounder, blowfish, filefish, sturgeon, catfish etc.

For use in accordance with embodiment of the present invention, linalool can be readily obtained from commercial sources; the most common source of linalool at present is linalool that has been synthesized from alpha-pinene or alpha-terpineol, which normally have been extracted from pine trees, although linalool can be obtained by conventional extraction techniques from a variety of natural sources, and if necessary to obtain a suitably high concentration of linalool (e.g. 90-100% linalool), further purified and/or concentrated. Preferably, the extracts from such natural sources contain at least 5 wt. % linalool before such purification/concentration steps. The following is a partial list of such sources: Allamanda cathartica linn flower oil, Artemisia santolina schrenk oil, Basil absolute sweet, Basil oil sweet, Basil oil var. glabratumBay leaf oil anise, Bay leaf oil clove, Bergamot mint oil, Bergamot oil, Bois de rose leaf oil, Bois de rose oil, Cananga oil, Cardamom seed oil, Carrot seed oil, Carrot weed oil, Cascarilla bark oil, Champaca absolute (michelia alba dc.), Champaca concrete, Clary sage oil, Coriander seed oil, Couroupita guianensis aubl. flower oil, Croton cajucara benth. leaf oil, Dialium guineense wild. fruit oil, Geranium leaf oil, Geranium oil, Glycosmis pentaphylla (cor.) seed oil, Ho leaf oil, Ho wood oil, Jasmin absolute concrete, Jasmin oil, Jasmin sambac absolute, Laurel bark oil, Laurel stem oil, Laurel wood oil, Lavandin absolute grosso, Lavandin oil, lavender flower oil, lavender oil, lavender stem oil, lecythis usitata miers. var. paraensis (ducke) r. kunth. flower oil, Mandarin oil, Marjoram oil, Myrtle oil, Narcissus absolute, Neroli bigarde oil, Neroli oil, Orange oil terpeneless, Orange flower absolute, Petitgrain bergamot oil, Petitgrain bigarade oil, Petitgrain mandarin oil, Petitgrain oil, Petitgrain sweet oil, Rosemary oil, Sage oil, Skimmia laureola oil, Snake root oil, Thyme absolute, Thyme oil, Ylang ylang oil, Yuzu peel oil. Linalool may also be synthesized, although it will be appreciated that in so-called “organic” applications will be preferable to use linalool obtained from a natural source. If the extract from the plant source has a sufficiently high linalool concentration, e.g. at least 80%, it may be used without further purification and/or concentration.

In accordance with embodiments of the present invention, linalool can be used in pure form or in a mixture. Such a mixture can be a suspension or emulsion of the linalool in water or can be a mixture in which the linalool is dissolved or suspended in an appropriate solvent or mixture of solvents. Examples of such solvents include alcohols such as ethanol. The linalool may be synthetic, or it may be isolated from a natural source. In some instances, the linalool may be provided as part of a plant extract, provided the linalool concentration within the extract is sufficiently high. An example of such an extract is Ho Wood Oil, an oil obtained by steam distillation of the wood of the camphor tree (Cinnamomum camphora) and which contains 80 wt. % or higher linalool.

In some embodiments, linalool may be used in the form of a composition which includes a surfactant. This surfactant can be any commercially available surfactant having suitable properties, such as ethoxylated castor oil. Examples of such surfactants are (1) anionic surfactants, such as (a) metal or amine salts of fatty acid, e.g. sodium stearate, (b) sulfates and ether sulphates, such as of alkyl or aryl chains, which can be ethoxylated, reacted with sulphuric acid and neutralised with metal or amine cations, e.g. sodium lauryl sulphate, (c) sulfonates, such as alkyl or aryl chains reacted with sulphonic acid and neutralised with metal or amine cations, e.g. sodium dodecylbenzenesulphonate; (2) nonionic surfactants, such as (a) ethoxylated alcohols and alkylphenols, such as alcohols attached to alkyl or aryl chains, of natural or synthetic origin, that have undergone degrees of ethoxylation, e.g. ethoxylated (20EO) hydrogenated castor oil or nonylphenol 9EO ethoxylate; (b) fatty acid esters, (c) fatty acid amides, (d) alkyl polysaccharides and (e) silicone ethoxylates; (3) cationic surfactants such as linear alkyl-amines and alkyl-ammoniums with or without aryl groups attached; and (4) amphoteric or zwitterionic surfactants such as betaines; as well as mixtures, blends and modifications therefore as will be appreciated by those skilled in the art. Further reference to the diverse range of possible surfactants can be found in standard reference books used by those skilled in the art, such as International Cosmetic Ingredient Dictionary and Handbook, 4-Volume Set, Twelfth Edition 2008, Personal Care Products Council. In some embodiments, the composition may contain a solvent such as propylene glycol.

In addition, linalool can be combined with one or more alternative food-grade aquatic sedative anaesthetic and/or euthanising agents. For example, a composition which includes linalool in combination with one more of eugenol, iso-eugenol, ethyl salicylate and methyl salicylate can be formulated.

The concentration of linalool employed in accordance with embodiments of the invention may vary, depending on the type of treatment to be be effected, viz. whether the organism is to be sedated, anaesthetised or euthanised. It will be appreciated that higher concentrations (50 mg linalool/l of water or even greater linalool concentrations) will normally be used where a deep anaesthetic or euthanising effect is desired to be achieved quickly. The concentration to be used will also depend on the time for which exposure to linalool is to be carried out (and vice versa); both of these variables will further depend on the type of fish to be treated (e.g. cold- or warm-water, fresh- or salt-water), the water temperature and the pH. The exact linalool concentrations and exposure times for a given species under given temperature and pH conditions may be determined empirically without undue experimentation.

It will also be understood that a progressive sedative to anaesthetic to euthanising effect can be induced by altering the time the organism is in contact with the linalool. Indeed, it is possible for the linalool to be used at concentrations of less than 10 ppm (eg 8 ppm or 5 ppm linalool in water containing the fish to be sedated), with a progressive sedative, anaesthetic and euthanising effect being induced by increased time exposure to the active solution.

The above concentration of about 5-8 ppm of the active compound can be employed when induction of the various graduated effects is not required quickly. It will be appreciated that the use of even lower or higher concentrations of the active compound(s) (100 ppm or above) is however not excluded.

The concentration of active agents will of course also vary when linalool is combined with one or more other agents such as eugenol, iso-eugenol, ethyl salicylate and methyl salicylate, again depending upon the effect to be induced. In some embodiments, the linalool is the predominant fish anesthetic in the water; in some embodiments, other anesthetics are not used, so that the water is substantially free of other fish anesthetics. In some embodiments, the concentration of each of eugenol, isoeugenol, ethyl salicylate, methyl salicylate or other fish anesthetics is at least three-fold, four-fold, five-fold, six-fold, seven-fold, eight-fold, nine-fold, or ten-fold less than the concentration of linalool.

Embodiments of the invention will now be illustrated with reference to the following Examples.

### EXAMPLE 1

A one year old koi fish of body length 11 cm and which appeared to be healthy was placed in a 4 liter tank containing water at 25° C. and in which the water was circulated by means of aeration. A liquid a mixture containing 1 wt. % synthetic linalool (1 wt. % linalool, 5 wt. % 14R, 2 wt. % AF80 and 92 wt. % water) was added to the tank and manually mixed with the water so that the concentration of linalool in the water was 50 ppm. This was regarding as the starting time of the experiment; thereafter, observations were made regularly to determine the behavior of the fish in water. This continued until no response to external stimulation and complete loss of equilibrium were observed, at which point the fish was transferred to a recovery tank containing fresh water. The fish was placed near the air pump, and the time until the fish again swam and responded normally to external stimulation was recorded.

Results: after 2.5 minutes exposure to linalool, the fish was in slight stress (the fish jerked often and surfaced often as if gasping for air). At 5.5 minutes, the fish exhibited partial loss of equilibrium and lying on the bottom of the tank. At 6.5 minutes, the fish exhibited complete loss of equilibrium. At 8 minutes the fish swam on its back. At 9 minutes the fish exhibited no response to external stimulation. At 9.5 minutes the fish was removed to a recovery tank with clean water. Thereafter it recovered slowly and after 45 minutes was swimming around in the tank and appeared to have resumed normal activity.

The results of this experiment indicate that the 1 wt. % linalool formulation, diluted to a concentration of 50 ppm linalool in water containing Koi fish, has an anesthetic effect thereupon.

### EXAMPLE 2

The experiment of Example 1 was repeated, using a one year old Koi fish of 12 cm body length, and adding the linalool formulation to a linalool concentration in the water of 75 ppm instead of 50 ppm. After 2 minutes of exposure to linalool the fish appeared to be in slight stress. At 5 5 minutes it exhibited partial loss of equilibrium and lying on the bottom of the tank. At 7 minutes it exhibited complete loss of equilibrium, and at 9 minutes it exhibited no response to external stimulation. At 20 minutes the fish was removed to a recovery tank with clean water. Thereafter it recovered slowly and after 40 minutes was swimming around in the tank and appeared to have resumed normal activity.

### EXAMPLE 3

The experiment of Example 1 was repeated, using a one year old Koi fish of 12.5 cm body length, and adding the linalool formulation to a linalool concentration in the water of 100 ppm instead of 50 ppm. After 2.5 minutes of exposure to linalool the fish exhibited partial loss of equilibrium, and at 4 minutes it exhibited complete loss of equilibrium. At 5.5 minutes it exhibited no response to external stimulation. At 6 minutes the fish was removed to a recovery tank with clean water. Thereafter it recovered slowly and after 30 minutes was swimming around in the tank and appeared to have resumed normal activity.

### EXAMPLE 4

The experiment of Example 1 was repeated, using a one year old goldfish of 8.5 cm body length. After 2 minutes of exposure to linalool the fish appeared to be in slight stress, at 5 minutes it exhibited partial loss of equilibrium, and at 6.5 minutes it exhibited complete loss of equilibrium. At 9 minutes it exhibited no response to external stimulation. At 9.5 minutes the fish was removed to a recovery tank with clean water. Thereafter it recovered slowly and after 35 minutes was swimming around in the tank and appeared to have resumed normal activity.

### EXAMPLE 5

The experiment of Example 1 was repeated, using a one year old goldfish of 8.5 cm body length, and adding the linalool formulation to a linalool concentration in the water of 75 ppm instead of 50 ppm. After 2 minutes of exposure to linalool the fish appeared to be in stress. At 2.5 minutes it exhibited partial loss of equilibrium. At 5 minutes it exhibited complete loss of equilibrium, and at 9 minutes it exhibited no response to external stimulation. At 20 minutes the fish was removed to a recovery tank with clean water. Thereafter it recovered slowly and after 35 minutes was swimming around in the tank and appeared to have resumed normal activity.

### EXAMPLE 6

The experiment of Example 1 was repeated, using a goldfish estimated to be one year old of 8 cm body length, and adding the linalool formulation to a linalool concentration in the water of 100 ppm instead of 50 ppm. After 1.5 minutes of exposure to linalool the fish exhibited partial loss of equilibrium, and 2 minutes it exhibited complete loss of equilibrium. At 3.5 minutes it exhibited no response to external stimulation. At 4 minutes the fish was removed to a recovery tank with clean water. Thereafter it recovered slowly and after 35 minutes was swimming around in the tank and appeared to have resumed normal activity.

### EXAMPLE 7

This experiment repeated the experiment of Example 1, using three goldfish (Carassius auratus auratus, average body length 12.5 cm) and five Ramirez fish (Mikrogeophagus ramirezi, body length 3.5-4 cm). All fishes appeared to be healthy. The goldfish tank held 4 liters of water and the tank holding the Ramirez fish one liter.

All fish were observed for a further three days and no mortality occurred.

### EXAMPLE 8

16 healthy carp (Cyprinus carpio), average weight of 150 g, maintained in 6 liter tanks, were each exposed to 75 ppm of linalool (using the same formulation described in Example 1, 45 g of which were mixed into each treatment tank) for varying amounts of time. Pairs of fish were introduced to linalool simultaneously and then removed to recovery tanks at various times: 3, 8, 12, 15, 20, 30, 40 and 50 minutes after introduction, and each fish's recovery was observed over the course of two consecutive days. Complete anesthesia was achieved after 5 minutes of exposure. Results are shown in the table below.

| | Fish no. | | | | | | | | | | | | | | | |
|---|---|---|---|---|---|---|---|---|---|---|---|---|---|---|---|---|
| time | 1 | 2 | 3 | 4 | 5 | 6 | 7 | 8 | 9 | 10 | 11 | 12 | 13 | 14 | 15 | 16 |
| 0 | Introduction of linalool | | | | | | | | | | | | | | | |
| 1 | Total loss of equilibrium | | | | | | | | | | | | | | | |
| 3 | RT | RT | Total loss of reactivity to all but strong external stimuli | | | | | | | | | | | | | |
| 4 | | | slow but regular opercular rate | | | | | | | | | | | | | |
| 5 | Loss of reflex reactivity (stage 5 anesthesia) | | | | | | | | | | | | | | | |
| 8 | | | RT | RT | | | | | | | | | | | | |
| 10 | | | | | opercular movements slow and irregular | | | | | | | | | | | |
| 12 | | | | | RT | RT | | | | | | | | | | |
| 13 | TR | TR | | | | | | | | | | | | | | |
| 15 | | | PR | | | | RT | RT | | | | | | | | |
| 16 | | | TR | PR | | | | | | | | | | | | |
| 17 | | | | TR | | | | | | | | | | | | |
| 19 | | | | | PR | | | | | | | | | | | |
| 20 | | | | | | PR | | | RT | RT | | | | | | |
| 21 | | | | | TR | | | | | | | | | | | |
| 22 | | | | | | TR | | | | | | | | | | |
| 24 | | | | | | | PR | | | | | | | | | |
| 25 | | | | | | | TR | | | | | | | | | |
| 27 | | | | | | | | | PR | PR | | | | | | |
| 28 | | | | | | | | PR | TR | TR | | | | | | |
| 29 | | | | | | | | TR | | | | | | | | |
| 30 | | | | | | | | | | | RT | RT | | | | |
| 38 | | | | | | | | | | | PR | | | | | |
| 39 | | | | | | | | | | | TR | PR | | | | |
| 40 | | | | | | | | | | | | TR | RT | RT | | |
| 50 | | | | | | | | | | | | | | | RT | RT |
| 51 | | | | | | | | | | | | | | | | |
| 70 | | | | | | | | | | | | | TR | | | |
| 77 | | | | | | | | | | | | | | PR | | |
| 78 | | | | | | | | | | | | | | TR | | |

Time is given in minutes;
RT = removed to recovery tank;
PR = partial recovery;
TR = total recovery.
Fish nos. 15 and 16 never recovered.

### EXAMPLE 9

This example illustrates the effects of Linalool on Goldfish (cold water fish). The experiment was performed in a four liter tank with three 10-month old goldfish of respective weights 32 g, 30 g and 34 g and respective body lengths of 12.5 cm, 11.5 cm and 11 cm. The water temperature was 16° C. and the pH was 7. A formulation containing 1% Linalool was added to the water to a concentration of 50 ppm linalool in the water. The fish became relaxed 5 minutes after introduction of the linalool, and after 6 5 minutes there fish were completely anesthetized. After 20 minutes of exposure to linalool, the fish were removed to a recovery tank with fresh water. 15 minutes thereafter the fish had resumed normal behavior.

### EXAMPLE 10

This experiment was performed in a four liter tank using three eight-month old goldfish of respective weights 27 g, 25 and 25.5 g and respective body lengths of 8.5 cm, 9 cm and 10 cm. The water temperature was 16° C. and the pH was 7. The same linalool-containing formulation as in Example 9 was to the water in the tank, this time to a linalool concentration in the water of 75 ppm. The fish became relaxed 2 5 minutes after addition of linalool, and at 5 minutes the fish were completely anesthetized. At 20 minutes the fish were removed in to a recovery tank with fresh water; after 35 minutes in the recovery tank the fish had resumed normal behavior.

### EXAMPLE 11

The experiment of example 9 was repeated, but this time the concentration of the linalool after mixing in the tank was 100 ppm. The three 8-month old goldfish were or respective weights 26 g, 27.5 g and 25 g and respective body lengths 12 cm, 11.5 cm and 12.5 cm. The fish became relaxed 1.5 minutes after addition of linalool, at 2 minutes the fish were completely anesthetized. At 4 minutes the fish were removed to a recovery tank with fresh water. After 20 minutes in the recovery tank the fish had resumed normal behavior.

### EXAMPLE 12

The experiment of example 9 was repeated, this time using five nine-month old Ramirez fish (M. ramirezi) in a one-liter tank. The respective weights were 0.8 g, 0.75 g, 0.7 g, 0.85 g and 0.75 g and the respective body lengths were 3.5 cm, 3.5 cm, 3 cm, 4 cm and 3 cm. The water temperature was 26° C. and the pH was 7. The fish became relaxed 2 minutes after addition of linalool, and after 5 minutes the fish were completely anesthetized. After 10 minutes the fish were removed to a recovery tank with fresh water. After 5 minutes in the recovery tank the fish had resumed normal behavior.

### EXAMPLE 13

To a 100 liter tank containing 462 10-month old carp fish (Cyprinus carpio) of average weight 70 g and average body length 10 cm, having water at 25° C. at pH 7 was added a formulation containing 1% linalool. The formulation was added to the water to a linalool concentration of 75 ppm in the water. The fish became relaxed 3 minutes after addition of linalool and after 5 minutes were completely anesthetized. After 15 minutes the fish were removed to a recovery tank with fresh water. After 20 minutes in the recovery tank the fish resumed normal behavior.

### EXAMPLE 14

To a 20 liter tank containing eight 2.5-year old carp fish of average weight 1 kg and average body length 50 cm in water at 20° C. and pH 7 was added a formulation containing 1% linalool. The formulation was added to the water to a linalool concentration of 100 ppm in the water. The fish became relaxed 2 minutes after addition of linalool and after 3 minutes were completely anesthetized. At 3 minutes the fish were removed to a recovery tank with fresh water. After 7 minutes in the recovery tank the fish resumed normal behavior.

### EXAMPLE 15

To a 20 liter tank containing eight 1.8-year old carp fish of average weight 250 g and body length 21-24 cm in water at 20° C. and pH 7 was added a formulation containing 1% linalool. The formulation was added to the water to a linalool concentration of 50 ppm in the water. The fish became relaxed 3 minutes after addition of linalool and after 7 minutes were completely anesthetized. At 17 minutes the fish were removed to a recovery tank with fresh water. After 29 minutes in the recovery tank the fish resumed normal behavior.

### EXAMPLE 16

To a 20 liter tank containing eight 2.2-year old carp fish of average weight 380 g and body length 30-32 cm in water at 20° C. and pH 7 was added a formulation containing 1% linalool. The formulation was added to the water to a linalool concentration of 100 ppm in the water. The fish became relaxed 2 minutes after addition of linalool and after 3 minutes were completely anesthetized. At 3 minutes the fish were removed to a recovery tank with fresh water. At 17 minutes in the recovery tank the fish resumed normal behavior.

### EXAMPLE 17

To a one liter tank containing five guppy fingerlings 21-30 days old of weight 0.010-0.014 g and body length 0.5 cm in water at 30° C. and pH 7 was added a formulation containing 92.5% linalool. The formulation was added to the water to a linalool concentration of 50 ppm in the water. The fish became relaxed 5 minutes after addition of linalool and after 7 minutes were completely anesthetized. At 10 minutes the fish were removed to a recovery tank with fresh water. After two minutes in the recovery tank the fish resumed normal behavior.

### EXAMPLE 18

To a four liter tank containing three one-year old Koi fish of respective weights 30 g, 29 g and 31 g and respective body lengths 11 cm, 12 cm and 12.5 cm in water at 21° C. and pH 7 was added a formulation containing 1% linalool. The formulation was added to the water to a linalool concentration of 50 ppm in the water. The fish became relaxed 6 minutes after addition of linalool and after 9 minutes were completely anesthetized. At 9 5 minutes the fish were removed to a recovery tank with fresh water. After 40 minutes in the recovery tank the fish resumed normal behavior.

### EXAMPLE 19

To a four liter tank containing eight 10-month old catfish (Clarias gariepinus) of respective weights 31 g, 14 g, 24 g and 9 g and respective body lengths 17 cm, 13 cm, 16 cm and 10 cm in water at 31° C. and pH 7 was added a formulation containing 1% linalool. The formulation was added to the water to a linalool concentration of 100 ppm in the water. The fish became relaxed 2 5 minutes after addition of linalool and after 4 minutes were completely anesthetized. At 5.5 minutes the fish were removed to a recovery tank with fresh water. After 7 minutes in the recovery tank the fish resumed normal behavior.

### EXAMPLE 20

The experiment of Example 1 was repeated, using two one-year old goldfish of respective body lengths 8.5 and 7.5 cm and respective weights of 17 and 15 g, and adding the a natural source of linalool (Ho Wood oil) to a concentration of 75 ppm linalool instead of 50 ppm. After two minutes of exposure to linalool the fish exhibited partial loss of equilibrium, and after 3 minutes they exhibited complete loss of equilibrium. At 3.5 minutes they exhibited no response to external stimulation. At 4 minutes the fish were removed to a recovery tank with clean water. After 5 minutes in the recovery tank they swam around and appeared to have resumed normal activity.

### EXAMPLE 21

The experiment of Example 21 was repeated, using two one-year old goldfish of respective body lengths 9 and 8 cm and respective weights 19 and 17.5 g, but adding Ho Wood oil to a linalool concentration of 50 ppm in the water. After seven minutes of exposure to linalool the fish appeared to exhibited partial loss of equilibrium, and after 15 minutes they exhibited complete loss of equilibrium. At 16 minutes they exhibited no response to external stimulation. At 16.5 minutes the fish were removed to a recovery tank with clean water. After 4 minutes in the recovery tank they swam around and appeared to have resumed normal activity.

### EXAMPLE 22

The experiment of Example 21 was repeated, using four 10-month old Guppy fish of respective body lengths 3, 3.5, 4 and 3 cm, and adding the Ho Wood oil to a linalool concentration of 50 ppm in the water. After 6 minutes of exposure to linalool the fish appeared to exhibit partial loss of equilibrium, and after 13 minutes they exhibited complete loss of equilibrium. At 14 minutes they exhibited no response to external stimulation. At 14.5 minutes the fish were removed to a recovery tank with clean water. After two minutes in the recovery tank they swam around and appeared to have resumed normal activity.

### EXAMPLE 23

The experiment of Example 21 was repeated, using four 10-month old Guppy fish of respective body lengths 4, 3.5, 3.5 and 3 cm, and adding the Ho Wood oil to a linalool concentration in the water of 75 ppm instead 50 ppm. After 1.5 minutes of exposure to linalool the fish appeared to exhibit partial loss of equilibrium, and after 2.5 minutes they exhibited complete loss of equilibrium. At 3 minutes they exhibited no response to external stimulation. At 3.5 minutes the fish were removed to a recovery tank with clean water. After two minutes in the recovery tank they swam around and appeared to have resumed normal activity.

### EXAMPLE 24

The experiment of Example 21 was repeated, using four one-year old Ramirez fish of respective body lengths 3, 2.5, 3 and 3.5 cm, and adding the Ho Wood oil to a linalool concentration of 50 ppm in the water. After 1 minute of exposure to linalool the fish appeared to exhibit partial loss of equilibrium, and after 1.5 minutes they exhibited complete loss of equilibrium. At 2 minutes they exhibited no response to external stimulation. At 2.5 minutes the fish were removed to a recovery tank with clean water. After 7 minutes in the recovery tank they swam around and appeared to have resumed normal activity.

### EXAMPLE 25

The experiment of Example 21 was repeated, using four 1-year old Ramirez fish of respective body lengths 3, 3.5, 3.5 and 4 cm, and adding the Ho Wood oil to a linalool concentration in the water of 75 ppm instead 50 ppm. After 25 seconds of exposure to linalool the fish appeared to exhibit partial loss of equilibrium, and after 40 seconds they exhibited complete loss of equilibrium. At 1 minute they exhibited no response to external stimulation. At 1.5 minutes the fish were removed to a recovery tank with clean water. After 7 minutes in the recovery tank the fish swam around and appeared to have resumed normal activity.

### EXAMPLE 26

The experiment of Example 21 was repeated, using two one-year old Black neon Tetra fish (Hyphessobrycon herbertaxelrodi) of 5 and 4 cm body length respectively, and adding the Ho Wood oil to a linalool concentration of 50 ppm in the water. After 0.5 minute of exposure to linalool the fish appeared to exhibit partial loss of equilibrium, and after 1 minute they exhibited complete loss of equilibrium. At 1.5 minutes they exhibited no response to external stimulation. At 2 minutes the fish were removed to a recovery tank with clean water. After 3 minutes in the recovery tank the fish swam around and appeared to have resumed normal activity.

### EXAMPLE 27

The experiment of Example 21 was repeated, using two one-year old Black neon Tetra fish (Hyphessobrycon herbertaxelrodi) of 4.5 and 4 cm body length respectively, and adding the Ho Wood oil to a linalool concentration in the water of 75 ppm instead of 50 ppm. After 1.5 minutes of exposure to linalool the fish appeared to exhibit partial loss of equilibrium, and after 3.5 minutes they exhibited complete loss of equilibrium. At 4 minutes they exhibited no response to external stimulation. At 4.5 minutes the fish were removed to a recovery tank with clean water. After 2 minutes in the recovery tank the fish swim around and appeared to have resumed normal activity.

### EXAMPLE 28

Three cyprinus carpio of 140 g average weight, each in a tank of 20 L water at 25° C., were exposed to linalool, added to each tank in a formulation containing 20 wt. % linalool, 5 wt. % Alkamuls 14/R (a non-ionic surfactant added as an emulsifier), 70 wt. % propylene glycol and 5 wt. % ethanol to a linalool concentration of 30 ppm in the water. Specimens were observed every 30 minutes after addition of linalool. At 30 minutes after exposure, each of the fish were easy to handle, and remained so for the duration of the three-hour exposure. After removal to a tank of untreated water, each of the fish returned to their initial state within five minutes.

### EXAMPLE 29

2-Phenoxyethanol (2-PE) is used to sedate fish in vaccinations procedures for relatively long periods of time (3 hours). 35 common carp were placed in each of two 60-liter tanks containing water at 26° C. Into one tank was added linalool (in a formulation as described in Example 28) to a concentration of 40 ppm; into the other tank was added 2-PE to a concentration of 200 ppm. Sedation of fish was determined by observation of loss of upright position, operculum movement and response to stimulus (handling). Total exposure time was five hours, at the end of which the fish were transferred 60-liter recovery tanks containing untreated water. The following results were observed:

Number of fish exhibiting loss of upright position

| Time | Linalool | 2-phenoxyethanol | Notes |
|---|---|---|---|
| 10:30 | 0 | 0 | Aeration |
| (start) | | | |
| 11:00 | 2 | 1 | No aeration |
| 11:40 | 9 | 2 | No aeration |
| 12:35 | 16 | 2 | No aeration |
| 13:35 | 17 | 2 | No aeration |
| 14:30 | 20 | 2 | Aeration |
| 15:30 | 16 | None | Aeration |
| Transfer to | All alive | All alive | Aeration |
| recovery tank | | | |

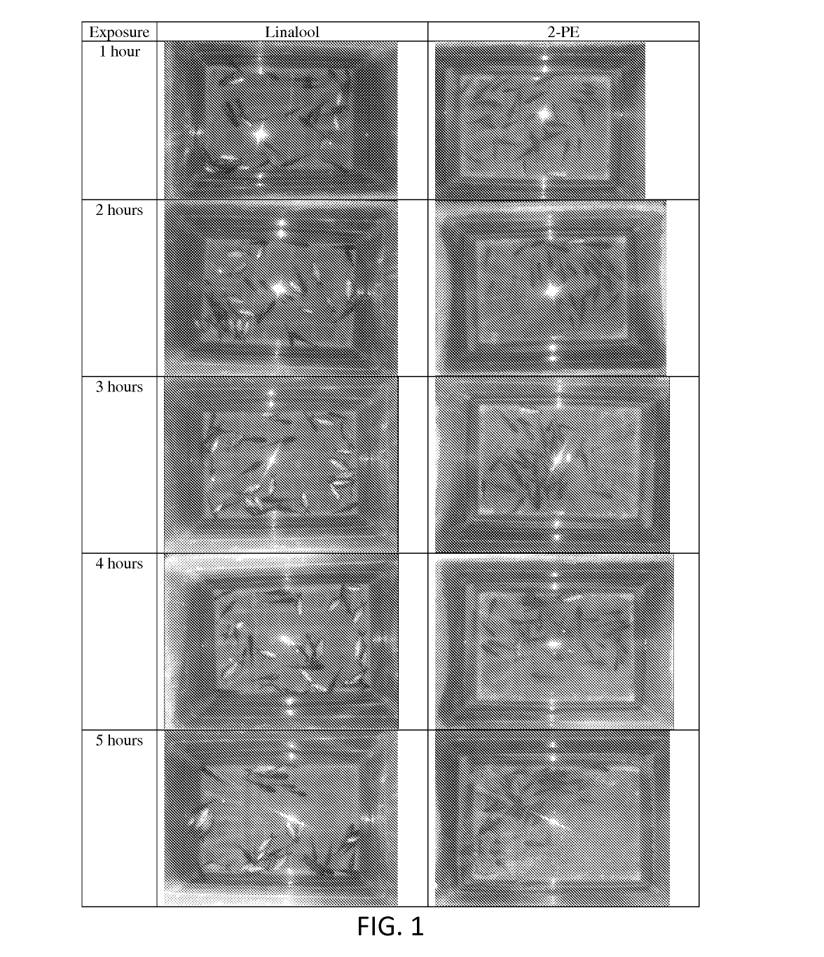

The reflex response of the linalool-exposed fish was reduced from the beginning of the trial and remained at the same level of reflex throughout the 5 hours of exposure in the water bath. In contrast, in the fish exposed to 2-phenyoxyethanol, the reflex response was reduced substantially from the beginning of the trial and increased with the passage of time until the end of the trial. In particular, in both treatments fish did not respond to external stimulation during the first 3 hours, and the sedation levels enabled handling of the fish. Between 3 and 5 hours of exposure, the sedative effect of 2-PE was reduced but that of linalool was maintained; but in contrast to 2-PE, linalool-exposed fish did not completely lose reflex reactivity. After placement in a recovery tank, all fish recovered quickly in few minutes and did not show signs of stress. FIG. 1 presents a series photographs of the tanks containing the linalool- and 2-PE-treated fish, taken from the top of the tanks, presented side-by-side. Fish swimming upright appear dark; fish on their sides appear light.

Persons skilled in the art will appreciate that the methods and compositions disclosed herein are useful in a variety of contexts. For example, the methods and compositions can be employed in the harvesting of aquatic organisms for ultimate human consumption, for instance with organisms such as fish which otherwise struggle violently to avoid capture, which has a significant impact on the post-mortem quality of the tissue: when sedated, anaesthetised or euthanised, this struggling is reduced, if not eliminated altogether. Further, the method may be practiced in a manner such that the residual concentration of the active agent in the tissue of the organism following harvesting will be very low and will therefore not detract from the suitability of the flesh for human consumption.

A further application of the sedating, anaesthetising or euthanising methods and compositions is in the transportation of live aquatic organisms. This is once again particularly the case with fish which are to be transported live to overseas markets and where the natural undamaged appearance of the fish is critical to the market price obtained.

1. A method of sedating, anaesthetising or euthanising an aquatic organism comprising the step of contacting said organism with a compound of the formula 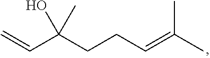
viz. linalool, wherein (a) said contacting occurs while said aquatic organism is in water, (b) the concentration of linalool in said water is at least 5 ppm and not more than 500 ppm, (c) the pH of the water is at least 5.5 and not more than 8.5, and (d) said contacting is for a period of at least 1 minute and not more than 180 minutes.

2-23. (canceled)

24. The method according to claim 1, wherein said water is at a temperature in the range of either (a) 4° C. to 24° C. or (b) 24° C. to 30° C.

25-68. (canceled)

69. The method according to claim 1, wherein said aquatic organism is a member of the class Chondrichthyes or Superclass Osteichthyes.

70. The method according to claim 69, wherein said member of the class Chondrichthyes or Superclass Osteichthyes is selected from the group consisting of salmon, trout, char, ayu, carp, crucian carp, goldfish, roach, whitebait, eel, conger eel, sardine, flying fish, sea bass, sea bream, parrot bass, snapper, mackerel, horse mackerel, tuna, bonito, yellowtail, rockfish, fluke, sole, flounder, blowfish, filefish, sturgeon, catfish and koi.

71. The method according to claim 69, wherein said member of the class Chondrichthyes or Superclass Osteichthyesis a cold-water fish.

72. The method according to claim 69, wherein said member of the class Chondrichthyes or Superclass Osteichthyesis a warm-water fish.

73. The method of claim 1, further comprising reversing a state of sedation or anesthetization induced by method according by placing said aquatic organism in water which is substantially free of fish sedative or fish anesthetic materials.

74. The method of claim 1, further comprising placing said aquatic organism in water which contains linalool at a lower concentration than the linalool concentration used to induce said sedation or anesthesia, whereby to maintain a state of sedation or anesthesia induced by the method of claim 1.

75. The method of claim 1, further comprising harvesting said aquatic organism.

76. The method according to claim 75, wherein said aquatic organism is harvested while anesthetized or sedated.

77. The method according to claim 75, wherein after said sedating or anesthetizing but before said harvesting, said aquatic organism is placed in water which contains linalool at a lower concentration than the linalool concentration used to induce said sedation or anesthesia.

78. The method according to claim 77 wherein said water which contains linalool at a lower concentration than the linalool concentration used to induce said sedation or anesthesia which is substantially free of fish sedative or fish anesthetic materials.

79. The method of claim 1, further comprising transporting said aquatic organism in a state of sedation or anesthesia induced by the method of claim 1, wherein said aquatic organism is transported in water which contains linalool at a sufficient concentration to maintain said aquatic organism in said state of sedation or anesthesia.

80-83. (canceled)

84. A composition comprising, in admixture, a compound of the formula 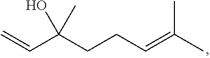
viz. linalool, and at least one solvent or surfactant, wherein the concentration said linalool in the composition is from 0.00005 wt. % to 93 wt. %.

85. (canceled)

86. A body of water containing at least one aquatic organism and linalool in a concentration of at least 5 ppm and not more than 500 ppm, wherein the pH of the water at least 5.5 and not more than 8.5.

87-102. (canceled)

103. A body of water according to claim 15, wherein said water is at a temperature in the range of either (a) 4° C. to 24° C. or (b) 24° C. to 30° C.

104-122. (canceled)

123. A body of water according to claim 86 wherein said body of water is enclosed in a man-made enclosure.

124. A body of water according to claim 123 wherein said man-made enclosure is sealed.

125. A body of water according to claim 86, wherein said aquatic organism is a member of the class Chondrichthyes or Superclass Osteichthyes.

126. A body of water according to claim 125, wherein said member of the class Chondrichthyes or Superclass Osteichthyesis selected from the group consisting of salmon, trout, char, ayu, carp, crucian carp, goldfish, roach, whitebait, eel, conger eel, sardine, flying fish, sea bass, sea bream, parrot bass, snapper, mackerel, horse mackerel, tuna, bonito, yellowtail, rockfish, fluke, sole, flounder, blowfish, filefish, sturgeon, catfish and koi.

127-130. (canceled)
